(19) United States
(12) Patent Application Publication
(10) Pub. No.: US 2004/0063094 A1
(43) Pub. Date: Apr. 1, 2004

(54) Mutant herpes simplex viruses and uses thereof

(75) Inventors: Robert Stuart Coffin, London (GB); David Seymour Latchman, London (GB)

Correspondence Address: NIXON & VANDERHYE, PC, 1100 N GLEBE ROAD, 8TH FLOOR, ARLINGTON, VA, 22201-4714, US

(73) Assignee: BIOVEX LIMITED, London (GB)

(21) Appl. No.: 10/668,164

(22) Filed: Sep. 24, 2003

Related U.S. Application Data
(63) Continuation of application No. 09/700,763, filed on Jan. 26, 2001, now abandoned.
Application No. 09/700,763 is a 371 of international application No. PCT/GB99/01598, filed on May 20, 1999.

(30) Foreign Application Priority Data
May 20, 1998 (GB) 9810904.4

(51) Int. Cl. C12Q 1/70; C12N 7/00; C12N 15/869

(52) U.S. Cl. 435/5; 435/456; 435/235.1

(57) ABSTRACT

A herpes simplex virus (HSV) comprising (i) an HSV LAT sequence inserted into an essential gene of the HSV; and (ii) a deletion in the endogenous LAT region of the HSV strain. The HSV of the invention can be used in the treatment of disorders of, or injuries to, the nervous system of a mammal.

### FIELD OF THE INVENTION

The present invention relates to mutant herpes simplex viruses comprising elements of the HSV latency associated transcript (LAT) region inserted into an essential gene and a deletion in the corresponding sequences of the endogenous LAT region. It also relates to the use of such mutant herpes simplex viruses in gene therapy and in methods of assaying for gene function.

### BACKGROUND TO THE INVENTION

Herpes simplex virus (HSV) has often been suggested as a suitable vector for the nervous system as it is neurotrophic and able to remain in neurons for the lifetime of the cell. However wild type HSV is highly pathogenic and must, like most viral vectors, be disabled in some way. The pathogenic effects of HSV result from lytic infection with the virus and therefore the use of HSV as a vector requires the development of strains carrying mutations that disrupt the lytic cycle whilst allowing the establishment of asymptomatic latent infections.

Herpes simplex virus (HSV) has often been proposed as a gene delivery vector for cells of both neuronal and non-neuronal origin. HSV may be particularly appropriate for gene delivery to the nervous system as it can naturally enter a life long latent state in these cells, and thus offers the possibility of a long term therapeutic effect if the expression of potentially therapeutic genes could be maintained throughout latency. By virtue of the large genome size of HSV, large DNA insertions can be made into the genome allowing the expression of multiple genes, which might be important for the therapy of particular diseases. However, before these particular advantages can be exploited the virus needs to be disabled both to prevent replication and reduce cytotoxicity, and promoter systems developed which allow the expression of inserted genes during latency.

For the production of an HSV vector strain, combinations of essential and non-essential genes can be removed from the genome so that the virus is nonpathogenic and minimally cytotoxic. Vector viruses are often produced by the deletion of one or other or both of the two essential immediate early genes ICP4 and ICP27. These require growth on cell lines expressing the deleted genes. Further deletions can be made to reduce cytotoxicity. For the production of viruses which allow gene expression during latency, promoters must be designed which allow gene expression to continue during this time, and this has proved to be a considerable challenge in the field of HSV vector development. However, we and others have found that a number of different promoter systems each incorporating different elements of the HSV latency associated transcript (LAT) region do give gene expression during latency to various levels of efficiency. These either use one or other of the LAT promoters (LAP1 or LAP2; Goins et al., 1994) to drive directly gene expression during latency, or DNA fragments derived from the LAT region to confer a long term activity on individual or pairs of promoters. This element, referred to herein as LAT P2 (including LAP2 and other upstream sequences; (nts 118866-112019-GenBank HE1CG)), has been shown subsequently to act not as a true promoter but instead to confer long term activity on heterologous promoters placed near to it, these promoters not being active during latency when used on their own.

### SUMMARY OF THE INVENTION

The present invention is based on our finding that the stability of HSV mutant genomes incorporating promoter systems based on LAT elements is reduced dramatically when the LAT-based promoter constructs are inserted into an essential IE gene region but not a non-essential gene region of the HSV genome such as US5 which is further removed from the endogenous LAT regions. The invention provides viruses which can be stably propagated which is not otherwise possible when such promoter constructs are inserted into an essential IE gene.

The present invention seeks to overcome the reduction in the stability of the resultant modified HSV genomes by the deletion of endogenous LAT sequences corresponding to those present in the promoter constructs which have inserted into an essential IE gene.

In particular, we have found that LAT-based promoters inserted into the IE essential IE genes (ICP4 and/or ICP27) without the deletion of corresponding elements in the endogenous LAT region can be re-arranged by homologous recombination during propagation of such mutant viruses on cell lines expressing complementing genes. This often results in the deletion of the inserted heterologous gene and also other sequences from the virus. However, if elements corresponding to the LAT sequences which have been inserted into ICP4 and, or ICP27 are deleted from the endogenous LAT regions, no re-arrangements can be detected and such viruses can be propagated in a stable fashion. The invention thus relates to viruses with insertions including elements of the LAT region into essential gene loci, preferably essential IE gene loci, corresponding elements having been deleted from the endogenous LAT regions of the virus. These viruses can be propagated in a stable fashion, such stable propagation not otherwise being possible.

Accordingly, the present invention provides a herpes simplex virus (HSV) comprising:

(i) an HSV LAT sequence inserted into an essential gene of the HSV; and

(ii) a deletion in the endogenous LAT regions of the HSV.

Preferably the deletion comprises at least some of the sequences present in the inserted HSV LAT sequence, preferably at least 50% of the sequences present in the inserted LAT sequence, more preferably at least 75%, most preferably at least all of sequences present in the inserted LAT sequence.

The essential gene into which the LAT sequence has been inserted preferably comprises a deletion, for example a deletion in the coding region and/or endogenous regulatory sequences of an essential gene. Insertions into essential IE genes are preferred.

The herpes simplex viruses of the invention can be used, for example, for delivering therapeutic genes in methods of treatment of diseases of, or injuries to, the nervous system, including Parkinson's disease, spinal injury or strokes, or diseases of the eye., heart or skeletal muscles, or malignancies. The present invention also relates to methods for studying the function of genes in mammalian cells, for example in identifying genes complementing cellular dysfunctions, or studying the effect of expressing mutant genes in wild-type or mutant mammalian cells. The methods of the present invention may be used in particular for the functional study of genes implicated in disease.

The invention further provides an HSV of the invention which carries a heterologous gene. The term heterologous gene is intended to embrace any gene not found in the HSV genome. The heterologous gene may be any allelic variant of a wild-type gene, or it may be a mutant gene. Heterologous genes are preferably operably linked to a control sequence permitting expression of said heterologous gene in mammalian cells, preferably cells of the central or peripheral nervous system, or cells of the eye, heart or skeletal muscle, more preferably cells of the central or peripheral nervous system. The HSV of the invention may thus be used to deliver a heterologous gene to a mammalian cell where it will be expressed. Such vectors are useful in a variety of applications, for example, in gene therapy, or in vitro assay methods or for the study of HSV gene regulation.

The heterologous gene preferably encodes a polypeptide of therapeutic use, including polypeptides that are cytotoxic or capable of converting a precursor prodrug into a cytotoxic compound.

The invention further provides herpes simplex viruses of the invention, carrying a heterologous gene, for use in the treatment of humans and animals. For example, such viruses may be used in the treatment of diseases of, or injury to, the nervous system,

including Parkinson's disease, spinal injury or strokes or disease of the eye, heart or skeletal muscle, or malignancies.

The HSV of the present invention may also be used in methods for studying the function of genes in mammalian cells, for example in identifying genes complementing cellular dysfunctions, or studying the effect of expressing mutant genes in wild-type or mutant mammalian cells. The methods of the present invention may be used in particular for the functional study of genes implicated in disease.

The invention also provides a method for producing a herpes simplex virus of the invention, said method comprising:

(i) inserting a HSV LAT sequence into an essential gene of the virus: and

(ii) deleting at least part of the LAT region of the virus.

Preferably the deletion in the LAT region of the HSV comprises at least some or more preferably all of the endogenous sequences corresponding to those present in the inserted HSV LAT sequence.

### DETAILED DESCRIPTION OF THE INVENTION

A. LAT sequences

LAT sequences are here defined to include all sequences from HSV1 strain 17+ between nucleotides 5.490 to 9.214 and 117,159 to 120.882 (GenBanK HE1CG) and homologous regions from other strains of HSV1 and all strains of HSV2.

The LAT P2 region is here defined as HSV1 nucleotides 118866 to 120219 (GenBank HEICG: from PstI-BstXI sites) in HSV1 strain 17− and fragments or derivatives of this region, including homologous regions of HSV2 and other strains of HSV1, which are capable of providing a long-term expression capability to promoters to which then are linked or which are themselves capable of driving long term gene expression.

B. Viral Strains

The herpes simplex viruses of the invention may be derived from, for example HSV1 or HSV2 strains, or derivatives thereof, preferably HSV1. Derivatives include inter-type recombinants containing DNA from HSV1 and HSV2 strains. Derivatives preferably have at least 70% sequence homology to either the HSV1 or HSV2 genomes, more preferably at least 80%, even more preferably at least 90 or 95%. Other derivatives which may be used to obtain the viruses of the present invention include strains that already have mutations in either ICP4 and/or ICP27, for example strain d120 which has a deletion in ICP4 (DeLuca et al., 1985). HSV strains have also been produced with deletions in ICP27, for example Reef Hardy and Sandri-Goldin, 1994 and Rice and Knipe, 1990 (strain d27-1). Strains no with deletions in both ICP4 and ICP27 are described in U.S. Pat. No. 5,658,724, and Samaniego et al. 1995 (strain d92). Use of these strains will reduce the number of steps required to produce the mutant HSV strains of the present invention.

The terminology used in describing the various HSV2 genes is as found in Coffin and Latchman, 1996.

C. Complementing Cell Lines

The virus of the invention is propagated on a cell line expressing an essential gene. Examples of cell lines that express ICP4 include E5 cells (DeLuca et al. 1985) or B4 cells (see Example 2), preferably B4 cells. Examples of cell lines that express ICP27 include V27 cells (Rice and Knipe. 1990), 2-2 cells (Smith et al., 1992) or B130/2 cells (see Example 1 and WO98/04726), preferably B130/2 cells.

Cell lines expressing essential genes can be produced by co-transfecting mammalian cells, for example the Vero or BHK cells, with a vector, preferably a plasmid vector, comprising a functional HSV essential gene capable of being expressed in said cells, and a vector, preferably a plasmid vector, encoding a selectable marker, for example neomycin resistance. Clones possessing the selectable marker are then screened further to determine which clones also express the functional essential gene, for example on the basis of their ability to support the growth of HSV strains lacking the essential gene, using methods known to those skilled in the art.

Cell lines which do not allow reversion of mutant HSV strain lacking a particular essential gene to a strain with the functional essential gene are produced as described above, ensuring that the vector comprising the functional essential gene does not, as far as possible, contain sequences that overlap with (i.e. are homologous to) sequences remaining in the mutant virus. Preferably, there is no overlap at all.

D. Methods of Mutation

Essential genes may be rendered functionally inactive prior to insertion of the LAT sequences by several techniques well known in the art. For example, they may be rendered functionally inactive by deletions, substitutions or insertions, preferably by deletion. Deletions may remove portions of the genes or the entire gene. For example, deletion of only one nucleotide may be made, resulting in a frame shift. However, preferably larger deletions are made, for example at least 25%, more preferably at least 50% of the total coding and non-coding sequence (or alternatively, in absolute terms, at least 10 nucleotides, more preferably at least 100 nucleotides, most preferably, at least 1000 nucleotides). It is particularly preferred to remove the entire gene and some of the flanking sequences. Inserted sequences may include the LAT sequences described above and the heterologous genes described below.

Deletions will also be made to remove part or all of the LAT region of the HSV. These deletions may be carried out as described below.

Mutations are made in the herpes simplex viruses by homologous recombination methods well known to those skilled in the art. For example. HSV genomic DNA is transfected together with a vector, preferably a plasmid vector, comprising the mutated sequence flanked by homologous HSV sequences. The mutated sequence may comprise deletions, insertions or substitutions, all of which may be constructed by routine techniques. Insertions may include selectable marker genes, for example lacZ, for screening recombinant viruses by, for example, β-galactosidase activity.

E. Heterologous Genes and Promoters

The mutant HSV strains of the invention may be modified to carry a heterologous gene, that is to say a gene other than one present in the HSV genome. The term “heterologous gene” comprises any gene other than one present in the HSV genome. The heterologous gene may be any allelic variant of a wild-type gene, or it may be a mutant gene. The term “gene” is intended to cover nucleic acid sequences which are capable of being at least transcribed. Thus, sequences encoding mRNA, tRNA and rRNA are included within this definition. The sequences may be in the sense or antisense orientation with respect to the promoter. Antisense constructs can be used to inhibit the expression of a gene in a cell according to well-known techniques. Sequences encoding mRNA will optionally include some or all of 5′ and/or 3′ transcribed but untranslated flanking sequences naturally, or otherwise, associated with the translated coding sequence. It may optionally further include the associated transcriptional control sequences normally associated with the transcribed sequences, for example transcriptional stop signals, polyadenylation sites and downstream enhancer elements.

The heterologous gene may be inserted into the HSV genome by homologous recombination of HSV strains with, for example, plasmid vectors carrying the heterologous gene flanked by HSV sequences. The heterologous gene may be introduced into a suitable plasmid vector comprising HSV sequences using cloning techniques well-known in the art. The heterologous gene may be inserted into the HSV genome at any location provided that the virus can still be propagated. It is preferred that the heterologous gene is inserted into an essential gene, preferably ICP4 or ICP7.

The transcribed sequence of the heterologous gene is preferably operably linked to a control sequence permitting expression of the heterologous gene in mammalian cells, preferably cells of the central and peripheral nervous system. The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequence.

The control sequence comprises a promoter allowing expression of the heterologous gene and a signal for termination of transcription. The promoter is selected from promoters which are functional in mammalian preferably human, cells. The promoter may be derived from promoter sequences of eukaryotic genes. For example, it may be a promoter derived from the genome of a cell in which expression of the heterologous gene is to occur, preferably a cell of the mammalian central or peripheral nervous system. With respect to eukaryotic promoters, they may be promoters that function in a ubiquitous manner (such as promoters of β-actin, tubulin) or, alternatively, a tissue-specific manner (such as promoters of the genes for pyruvate kinase). They may also be promoters that respond to specific stimuli, for example promoters that bind steroid hormone receptors. Viral promoters may also be used, for example the Moloney murine leukaemia virus long terminal repeat (MMLV LTR) promoter or promoters of HSV genes.

The HSV LAT promoter, and promoters containing elements of the LAT promoter region, are especially preferred because there is the possibility of achieving long-term expression of heterologous genes during latency. In particular, an expression cassette consisting essentially of a LAT P2 region, which does not itself here act as a promoter, linked to a promoter and a heterologous gene in that order is especially preferred.

The term “long-term expression” is taken to mean expression of a heterologous gene in a cell infected with a herpes simplex virus of the invention even after the herpes simplex virus has entered latency. Preferably, this is for at least two weeks, more preferably at least one or two months after infection, even more preferably for the life-time of the cell.

The expression cassette may further comprise a second promoter and a second heterologous gene operably linked in that order to said HSV LAT P2 region and in the opposite orientation to the first promoter and first heterologous gene wherein said second promoter and second heterologous gene are the same as or different to the first promoter and first heterologous gene. Thus a pair of promoteriheterologous gene constructs in opposite orientations flank a single LAT P2 region allowing the long term expression of pairs of heterologous genes, which may be the same or different, driven by the same or different promoters. Furthermore, the product of the first heterologous gene may regulate the expression of the second heterologous gene (or vice-versa) under suitable physiological conditions.

The expression cassette can be constructed using routine cloning techniques known to persons skilled in the art (see, for example, Sambrook et al., 1989. Molecular Cloning—a laboratory manual; Cold Spring Harbor Press). Furthermore, the construction of particular HSV strains comprising such an expression cassette is described in the Examples.

The LAT P2 region is here defined as HSV1 nucleotides 118866 to 120219 (GenBank HEICG: from PstI-BstXI sites), fragments or derivatives of this region, including homologous regions of HSV2, which are capable of providing a long-term expression capability to promoters to which they are linked.

It may also be advantageous for the promoters to be inducible so that the levels of expression of the heterologous gene can be regulated during the life-time of the cell. Inducible means that the levels of expression obtained using the promoter can be regulated. For example, in a preferred embodiment where more than one heterologous gene is inserted into the HSV genome, one promoter would comprise a promoter responsive to the tet repressor/VP16 transcriptional activator fusion protein previously reported (Gossen and Bujard, 1992. Gossen et al, 1995), and driving the heterologous gene the expression of which is to be regulated. The second promoter would comprise a strong promoter (e.g. the CMV IE promoter) driving the expression of the tet repressor/VP16 fusion protein. Thus in this example expression of the first heterologous gene would depend on the presence or absence of tetracycline.

In addition, any of these promoters may be modified by the addition of further regulatory sequences, for example enhancer sequences (including elements of the LAT region). Chimeric promoters may also be used comprising sequence elements from two or more different promoters described above, for example an MMLV LTR/LAT fusion promoter (Lokensgard et al., 1994) or promoters comprising elements of the LAT region (see above).

The heterologous gene may encode, for example, proteins involved in the regulation of cell division, for example mitogenic growth factors including neurotrophic growth factors (such as brain-derived neurotrophic factor, glial cell derived neurotrophic factor, NGF, NT3, NT4 and NT5, GAP43), cytokines (such as α-, β- or γ-interferon, interleukins including IL-1, IL-2, tumour necrosis factor, or insulin-like growth factors I or II), protein kinases (such as NIAP kinase), protein phosphatases and cellular receptors for any of the above. The heterologous gene may also encode enzymes involved in cellular metabolic pathways, for example enzymes involved in amino acid biosynthesis or degradation (such as tyrosine hydroxylase), purine or pyrimidine biosynthesis or degradation, and the biosynthesis or degradation of neurotransmitters, such as dopamine, or protein involved in the regulation of such pathways, for example protein kinases and phosphatases. The heterologous gene may also encode transcription factors or proteins involved in their regulation, for example members of the Brn3 family (including Brn3a, Brn3b and Brn3c) or pocket proteins of the Rb family such as Rb or p107, membrane proteins (such as rhodopsin), structural proteins (such as dystrophin) or heat shock proteins such as hsp27, hsp65, hsp70 and hsp90.

Preferably, the heterologous gene encodes a polypeptide of therapeutic use, or whose function or lack of function may be important in a disease process. For example, of the proteins described above, tyrosine hydroxylase can be used in the treatment of Parkinson's disease, rhodopsin can be used in the treatment of eve disorders, dystrophin may be used to treat muscular dystrophy, and heat shock proteins can be used to treat disorders of the heart and brain associated with ischaemic stress. Polypeptides of therapeutic use may also include cytotoxic polypeptides such as ricin, or enzymes capable of converting a precursor prodrug into a cytotoxic compound for use in, for example, methods of virus-directed enzyme prodrug therapy or gene-directed enzyme prodrug therapy. In the latter case, it may be desirable to ensure that the enzyme has a suitable signal sequence for directing it to the cell surface, preferably a signal sequence that allows the enzyme to be exposed on the exterior of the cell surface whilst remaining anchored to cell membrane. Suitable enzymes include bacterial nitroreductase such as E. coli nitroreductase as disclosed in WO93/08288 or carboxypeptidase, especially carboxypeptidase CPG2 as disclosed in WO88/07378. Other enzymes may be found by reference to EP-A-415731. Suitable prodrugs include nitrogen mustard prodrugs and other compounds such as those described in WO88/07378, WO89/10140, WO90/0′-1729 and WO93/08288 which are incorporated herein by reference.

Heterologous genes may also encode antigenic poly-peptides for use as vaccines. Preferably such antigenic polypeptides are derived from pathogenic organisms, for example bacteria or viruses, or from tumours.

Heterologous genes may also include marker genes (for example encoding β-galactosidase or green fluorescent protein) or genes whose products regulate the expression of other genes (for example, transcriptional regulatory factors including the tet repressor/VP16 transcriptional activator fusion protein described above).

Gene therapy and other therapeutic applications may well require the administration of multiple genes. The expression of multiple genes may be advantageous for the treatment of a variety of conditions—e.g. using multiple neurotrophic factors. HSV is uniquely appropriate as it does not have the limited packaging capabilities of other viral vector systems. Thus multiple heterologous genes can be accommodated within its genome. There are, for example, at least two ways in which this could be achieved. For example, more than one heterologous gene and associated control sequences could be introduced into a particular HSV strain. It would also be possible to use pairs of promoters (the same or different promoters) facing in opposite orientations away from a centrally located LAT P2 element, these promoters each driving the expression of a heterologous gene (the same or different heterologous gene) as described above.

F. Administration

The mutant herpes simplex viruses of the present invention may thus be used to deliver therapeutic genes to a human or animal in need of treatment. Delivery of therapeutic genes using the mutant herpes simplex viruses of the invention may be used to treat for example, Parkinson's disease, disorders of the nervous system, spinal injury, strokes or malignancies, for example gliomas.

One method for administered gene therapy involves inserting the therapeutic gene into the genome of the mutant herpes simplex virus of the invention, as described above, and then combining the resultant recombinant virus with a pharmaceutically acceptable carrier or diluent to produce a pharmaceutical composition. Suitable carriers and diluents include isotonic saline solutions, for example phosphate-buffered saline. The composition may be formulated for parenteral, intramuscular, intravenous, subcutaneous, intraocular or transdermal administration.

The pharmaceutical composition is administered in such a way that the mutated virus containing the therapeutic gene for gene therapy, can be incorporated into cells at an appropriate area. For example, when the target of gene therapy is the central or peripheral nervous system, the composition could be administered in an area where synaptic terminals are located. The pharmaceutical composition is typically administered to the brain by stereotaxic inoculation. When the pharmaceutical composition is administered to the eye, sub-retinal injection is typically the technique used.

The amount of virus administered is in the range of from 10⁴ to 10¹⁰ pfu, preferably from 10⁵ to 10⁸ pfu, more preferably about 10⁶ to 10⁷ pfu. When injected, so typically 1 to 10 μl of virus in a pharmaceutically acceptable suitable carrier or diluent is administered.

The routes of administration and dosages described are intended only as a guide since a skilled practitioner will be able to determine readily the optimum route of administration and dosage for any particular patient and condition.

G. Assays Methodologies

The mutant herpes simplex viruses of the invention can also be used in methods of scientific research. Thus, a further aspect of the present invention relates to methods of assaying gene function in mammalian cells, either in vitro or in vivo. The function of a heterologous gene could be determined by a method comprising:

(a) introducing said heterologous gene into a mutant herpes simplex virus of the invention;

(b) introducing the resulting virus into a mammalian cell line, and

(c) determining the effect of expression of said heterologous gene in said mammalian cell-line.

For example, the cell-line may have a temperature-sensitive defect in cell division. When an HSV strain comprising a heterologous gene according to the invention is introduced into the defective cell-line and the cell-line grown at the restrictive temperature, a skilled person will easily be able to determine whether the heterologous gene can complement the defect in cell division. Similarly, other known techniques can be applied to determine if expression of the heterologous gene can correct an observable mutant phenotype in the mammalian cell-line.

This procedure can also be used to carry out systematic mutagenesis of a heterologous gene to ascertain which regions of the protein encoded by the gene are involved in restoring the mutant phenotype.

This method can also be used in animals, for example mice, carrying so-called “gene knock-outs”. A wild-type heterologous gene can be introduced into the animal using a mutant HSV strain of the invention and the effect on the animal determined using various behavioural, histochemical or biochemical assays known in the art. Alternatively, a mutant heterologous gene can be introduced into either a wild-type or “gene knock-out” animal to determine if disease-associated pathology is induced. An example of this is the use of genes encoding prions to induce Creutzfeld-Jacob and other prion-type diseases in the central nervous system of rodents. Other disease models may include those for Alzheimer's disease, motor neurone disease or Parkinson's disease.

Since it is possible to introduce at least two different heterologous genes into a cell due to the large capacity of the HSV genome, it will also be possible to study the interaction between two or more gene products.

Thus, the methods of the present invention may be used in particular for the functional study of genes implicated in disease.

The invention will be described with reference to the following Examples, which are intended to be illustrative only and not limiting.

### EXAMPLES

### Reference Example 1

### Preparation of an ICP4-Complementing Cell Line (B4).

A complementing cell line (B4) allowing growth of ICP4 deleted viruses was generated by co-transfection of plasmid pICP4 DNA with neomycin resistance-encoding plasmid pMamNeo (Invitrogen) into BHK cells and the selection of neomycin resistant clones. Plasmid pICP4 contains a DdeI-SphI fragment from the HSV1 genome (nucleotides 126,764-131,730), containing the ICP4 coding region and promoter, cloned between the EcoRV and SphI sites of pSP7′ (Promega)

A clone highly permissive for the growth of an HSV1 ICP4 deletion mutant (B4) was selected for virus growth.

### Reference Example 2

### Preparation of an ICP27-Complementing Cell Line (B130/2).

A complementing cell line (B130/2) allowing growth of ICP27 deleted viruses and having no overlap between the complementing sequences and the ICP27 deleted viruses above (and thus preventing repair of ICP27 by homologous recombination during virus growth) was generated by co-transfection of plasmid pSG130BS (Sekulovich et al. 1988) DNA with neomycin resistance-encoding plasmid pMamNeo (Invitrogen) into BHIK cells and the selection of neomycin resistant clones. A clone highly permissive for the growth of an HSV-1 ICP27 deletion mutant (B130/2) was selected for virus growth. PSG130BS carries a BamHI/SacI fragment from HSV1 (nucleotides 113322-115743) encoding the complete ICP27 coding sequence and part of UL55.

### Example 1

### HSV Strains in Which Promoters Containing LAT Sequences are Inserted so as to Delete ICP27, but Without Deletion in the Endogenous LAT Region, Cannot be Stably Propagated

(a) A cassette from plasmid pR20.5 consisting of an RSV/lacZ/pA sequence and a CMV/GFP/pA sequence in opposite back-to-back orientations and separated by an HSV LAT region sequence (nucleotides 118.866-120.219) was inserted into the ICP27 locus by homologous recombination with purified genomic HSV1 strain 17+ DNA by standard methods. The pR20.5 cassette was first inserted in both orientations into a plasmid containing ICP27 flanking regions (pΔ7), allowing the production of two viruses with an HSV LAT sequence in either the same or the opposite orientation to the LAT sequence in the nearby endogenous LAT region. These viruses are deleted for the entire ICP27 gene. The pR20.5 cassette can be excised from its pGEM5 (Promega) plasmid backbone with Srf1 as an oligonucleotide encoding SrfI was inserted on either side of the cassette. The RSV promoter was excised from pRc/RSV (Invitrogen), lacZ/pA from pCH110 (Pharmacia), CMV/pA from pcDNA3 (Invitrogen) and GFP from pEGFP-NI (Clontech) for the construction of plasmid pR20.5. pΔ77 was constructed by first subcloning an EcoRI-NotI fragment from HSV1 restriction fragment EcoRI B (nucleotides 110095-131534) which includes the ICP27 gene into pGEM5 (Promega) and deleting the ICP27 (UL54), UL55 and UL56 encoding MluI fragment by digestion with MluI and religation. The pR20.5 cassette was then inserted at the now unique MluI site.

Viruses were plaque purified by selection of green GFP expressing plaques under fluorescence microscopy, and stocks were prepared using B130/2/cells (described above in Reference Example 2). The resulting virus stocks (17+/pR20.5/27 and 17+/pR20/27rev) were unable to give a productive infection on BHK cells which do not complement the ICP27 deletion.

Stocks of the viruses were prepared by inoculating single plaques into a well of a six well plate, harvesting this stock and inoculating new six well plates. Stocks from these plates were then titrated onto B130/2 cells. The numbers of green (under fluorscence microscopy), blue (after X-gal staining to detect lacZ) and white plaques after each procedure was counted. For 17+/pR20.5/27 the GFP gene is between the inserted LAT sequence and the endogenous LAT region and for 17+/pR20/27rev the lacZ gene is between the inserted and endogenous LAT sequences.

Results

After growth of virus stocks and titration it was found that not all plaques now expressed both the GFP and lacZ genes. In the case of 17−/pR20.5/27 it was found that while all plaques expressed lacZ, approximately 2% no longer expressed GFP. This suggested homologous recombination between the inserted and endogenous LAT sequences, which are here in the same orientation, deleting the intervening sequences including GFP at a relatively low level during virus growth. With 17+/pR20/27rev, where inserted and the nearby endogenous LAT sequences are in opposite orientations with respect to each other, most plaques still expressed both lacZ and GFP. However approximately 1% did not express either lacZ or GFP or both inserted genes, suggesting that when in the reverse orientation with respect to each other, more complex recombinational mechanisms occur between inserted and endogenous LAT sequences which can result in the deletion of one or other or more rarely both inserted genes.

(b) A cassette consisting of a LAT sequence (DdeI-DdeI: nucleotides 118.180 to 118,768) followed by an MMLV LTR promoter from pJ4 (Morgenstern and Land 1990) separated by an approximately 700 bp plasmid spacer sequence (NdeI-Smal from pGEM3zf-Promega) followed by a lacZ/pA sequence from pCH110 (Pharmacia) was inserted into pA27 as in (a) above. This plasmid is called pR18/27. The LAT sequence is here in the same orientation in the nearby endogenous LAT region. A virus was constructed (17+/pR18/27) and stocks grouts up as in (a) above using B130/2 cells, here selecting for the expression of lacZ by X-gal staining. Stocks were again titred and numbers of blue and white ICP27 deleted plaques counted after X-gal staining.

Results

After two serial passages as described, approximately 3% of resulting plaques no longer expressed lacZ. This suggests that homologous recombination between inserted and endogenous LAT regions has led to the deletion of intervening sequences, including the lacZ gene.

### Example 2

### HSV Strains in Which Promoters Containing LAT Sequences are Inserted so as to Delete ICP4, but Without Deletion in the Endogenous LAT Region, Cannot be Stably Propagated

The pR20.5 cassette was inserted into the ICP4 locus of HSV1 with accompanying deletion of ICP4 by insertion of the pR20.5 cassette into ICP4 flanking regions (plasmid pΔ4) generating plasmid pR20.5/4 and homologous recombination together with purified genomic HSV1 strain 17+ DNA into B4 cells (produced as described in Reference Example 1). pA4 was consists of ICP4 flanking sequences nucleotides 123.459-126.774 (Sau3a-Sau3a) and nucleotides 131,730-134,792 (SphI-KpnI) in pSP72 (Promega) separated by XbaI and SalI sites derived from pSP72. pICP4 contains a DdeI-SphI fragment from HSV1 (nucleotides 126.764-131.730) cloned between the EcoRV and SphI sites of pSP72. The resulting virus (17−/pR20.5/4) was unable to grow on BHK cells which do not compliment the deletion in ICP4.

As before, stocks of the virus were prepared by inoculating single plaques into a single well of a six well plate, harvesting this stock and inoculating new six well plates. These stocks were then used for inoculation of 175 cm² flasks. Stocks from both plates and flasks were then titrated onto B4 cells. The numbers of green (under fluorscence microscopy), blue (after X-gal staining to detect lacZ) and white plaques after each procedure were again counted, in 17+/pR0.5/4 the site of insertion (ICP4) means that endogenous and inserted LAT sequences in opposite orientations are separated from each other by the GFP gene and also the coding sequences for ICP0. RL1 and ORFP, and also other less well characterised coding sequences. Deletion of ICP0, although probably not RL1 ORFP or other sequences. by homologous recombination between endogenous and inserted LAT sequences would be expected to result in a virus with significantly impaired growth characteristics as compared to IE1 containing viruses.

Results

Titres of virus stocks prepared from six well plates showed no detectable deletion of the lacZ or GFP genes. However when stocks which had now been serially passaged 3 times were titrated from the large flasks, small numbers of plaques which no longer expressed one or other or both genes could be detected, even though at no time were high MOI infections used to generate the stocks which had been titrated. Thus here approximately 0.5% of plaques no longer expressed either the GFP or lacZ gene, and approximately 0.1% no longer expressed either gene. It can be concluded that homologous recombination between the inserted LAT sequence in both copies of ICP4 and one or other or both copies of the endogenous LAT sequences present in the long repeat regions of the genome allowed recombinational mechanisms to occur which could allow the deletion of one or other or both inserted marker genes.

### Example 3

### HSV Strains in Which Promoters Containing LAT Sequences are Inserted into US5 can be Stably Propagated

The pR20.5 cassette was inserted into the US5 locus of HSV1 by insertion of the pR20.5 cassette into US5 flanking regions (plasmid pΔUS5) generating plasmid pR20.5/US5 followed by homologous recombination together with purified genomic HSV1 strain 17− DNA into BHK cells giving virus strain 17+/pR20.5/US5. Plasmid pΔUS5 was prepared by cloning a BamHI-EcoNI fragment (nucleotides 136.289 to 131.328) from HSV1, which includes the US5 coding region, into plasmid pAT153. pR20.5 was inserted into a unique SacI site at nucleotide 137,945 in the US5 gene. In 17+/pR20.5/US5 no essential gene is deleted and so cells complementing the deletion are not required.

As before, after plaque purification, stocks of the virus were prepared by inoculating single plaques into a single well of a six well plate, harvesting this stock and inoculating new six well plates. Serial passage was continued five times before inoculation of 175 cm² flasks. Stocks from both plates and flasks were titrated onto BHK cells and the numbers of green, blue and white plaques counted.

Results

In each case the virus titred from either six well plates or 175 cm² flasks showed no detectable deletion of the lacZ or GFP genes, even after serial passage.

### Conclusions from Examples 1 to 3

Insertion of LAT sequences into either the ICP27 or ICP4 locus of HSV results in HSV genomes which are unstable as they can be rearranged by homologous recombination between endogenous and inserted LAT sequences, even though deletion of intervening sequences might be expected to result in a virus with a growth disadvantage as compared to the background virus. Insertions of LAT sequences into the US5 locus on the other hand, which is at a position in the HSV genome further removed from the endogenous LAT sequences than either ICP27 or ICP4, does not result in a virus in which stable propagation is prevented.

### Example 4

### HSV Strains in Which Sequences from the LAT Region Corresponding to those Inserted Elsewhere in the Genome are Deleted can be Propagated in a Stable Fashion

(a) A virus was constructed in which LAT sequences corresponding to the LAT sequence in the pR20.5 cassette was deleted from both endogenous LAT regions before insertion of further sequences. This virus (17+/p2−) was constructed by the insertion of a CMV/lacZ cassette between two BstXI sites in the LAT region (BstXI sites cutting at nucleotides 120,217 and 120,406) and selection of lacZ expressing plaques by homologous recombination of plasmid pR19lacZ together with genomic HSV1 strain 17− DNA. The CMV/lacZ cassette together with LAT sequences (nucleotides 118,769 to 120,469) were then deleted by homologous recombination with a second plasmid (pΔP2) containing flanking regions so as delete the required sequences and selection of white (after X-gal staining) non-lacZ expressing plaques. pR19lacZ was constructed by insertion of a CMV/lacZ/polyA cassette into pNot3.5 between the BstXI sites. pNot3.5 consisting of a 3.5 kb NotI fragment from the LAT region (nucleotides 118,439 to 122,025) cloned into the NotI site of pGem5 (Promega). pΔP2 consists of the DdeI fragment (nucleotides 118,180 to 118,768) from the LAT region followed by the HpaI-NotI fragment (nts 120,470-129,095) cloned into pGem5 and separated by SacI, KpnI and AvaI polylinker derived sites. 17+/p2− was checked by Southern blotting to confirm deletion of both copies of the LAT sequences from the long repeat regions of the genome. The pR20.5 cassette was then inserted into the ICP27 locus using plasmid pR20.5 27 as in example 2. The resulting virus (17+/P2−/pR20.5/27) was unable to grow on BHK cells which do not complement the deletion in ICP27.

Stocks of the viruses were again prepared by inoculating single plaques into a well of a six well plate, harvesting this stock and inoculating new six well plates. Stocks from these were used to inoculate 175 cm² flasks and stocks from both were then titrated onto B130/2 cells. The numbers of green (under fluorscence microscopy), blue (after X-gal staining to detect lacZ) and white plaques after each procedure were again counted.

Results

After growth of virus stocks and titration it was found that all plaques now expressed lacZ genes when either virus stock was titrated out. This suggested homologous recombination between the inserted and endogenous LAT sequences could no longer occur thus allowing stable propagation of the virus.

(b) The pR20.5 cassette was also inserted into the ICP4 locus of 17+/p2− using plasmid pR20.5/4 as in Example 2. The resulting virus (17−P2−/pR20.5/4) was unable to grow on BHK cells, which do not complement the deletion in ICP4.

As before stocks of the virus were prepared by inoculating single plaques into a single well of a six well plate, harvesting this stock and inoculating new six well plates. Serial passage was continued five times before inoculation of 175 cm² flasks. Stocks from both plates and flasks were as before titrated onto B4/27/4 cells and numbers of green, blue and white plaques counted.

Results

Titres of virus stocks prepared from initial six well plates showed no detectable deletion of the lacZ or GFP genes. When stocks which had been serially passaged a further five times before the inoculation of large flasks were titred, only plaques which expressed both GFP and lacZ could be detected, with no plaques which only expressed one or other or neither gene observed. This suggested that prior deletion of endogenous LAT sequences corresponding to the sequences inserted in pR20.5 had again provided a virus with greater stability than a virus in which endogenous LAT sequences were not first deleted, thus allowing stable propagation of the virus.

### REFERENCES

Coffin R S, Latchman D S. Herpes simplex virus-based vectors. In: Latchman D S (ed). Genetic manipulation of the nervous system. Academic Press: London, 1996, pp 99-114.

Morgenstern J P and Land H. A series of mammalian expression vectors and characterisation of their expression of a reporter gene in stably and transiently transfected cells. NAR 1990; 18: 1068.

Reef Hardy, W and Sandri-Goldin R M. Herpes simplex virus inhibits host cell splicing and regulatory protein ICP27 is required for this effect J. Virol 1994: 68: 7790-7799.

DeLuca N A et al. J. Virol. 1985; 56: 558-570.

Sekulovich R E et al., 1988, J. Virol. 64; 3916-3926.

Lokensgard J R et al. J. Virol. 1994; 68: 7148-7158.

1. A herpes simplex virus (HSV) comprising:
(i) an HSV LAT sequence inserted into an essential gene of the HSV; and
(ii) a deletion in the endogenous LAT region of the HSV.

2. A virus according to claim 1 wherein the essential gene is an essential immediate early (IE) gene.

3. A virus according to claim 2 wherein the essential IE gene is ICP7.

4. A virus according to claim 2 wherein the essential IE gene is ICP4.

5. A virus according to any one of the preceding claims wherein the deletion comprises at least some of the sequences present in the inserted HSV LAT sequence.

6. A virus according to any one of the preceding claims wherein the deletion comprises at least 50% of the sequences present in the inserted LAT sequence.

7. A virus according to any one of the preceding claims wherein the deletion comprises at least 75% of the sequences present in the inserted LAT sequence.

8. A virus according to any one of the preceding claims wherein the deletion comprises at least all of the sequences present in the inserted LAT sequence.

9. A virus according to any one of the preceding claims wherein the LAT sequence consists essentially of nucleotides 118866 to 120219 and/or nucleotides 117159 to 118865 of HSV1 strain 17+ (GenBank HE1 CG), or the homologous sequences of another HSV strain.

10. A virus according to any one of the preceding claims wherein the essential gene comprises a deletion.

11. A virus according to any one of the preceding claims which is selected from an HSV1 strain, an HSV2 strain or derivatives thereof.

12. A virus according to claim 11 which is an HSV1 strain.

13. A virus according to any one of the preceding claims which carries at least one heterologous gene.

14. A virus according to claim 13 wherein said heterologous gene is operably linked to an/the inserted HSV LAT sequence.

15. A virus according to claim 13 or 14 wherein said heterologous gene is operably linked to a control sequence permitting expression of said heterologous gene in mammalian cells.

16. A virus according to claim 1 wherein said mammalian cell is a cell of the central or peripheral nervous system of a mammal.

17. A virus according to claim 15 wherein said mammalian cell is a cell of the eye, heart or skeletal muscle of a mammal.

18. A virus according to any one of claims 13 to 17 wherein said heterologous gene encodes a polypeptide of therapeutic use.

19. A virus according to claim 18 wherein said gene encodes a polypeptide which is cytotoxic.

20. A virus according to claim 18 wherein said gene encodes a polypeptide capable of converting a precursor prodrug into a cytotoxic compound.

21. A virus according to any one of claims 15 to 18 wherein the heterologous gene is selected from genes encoding proteins involved in the regulation of cell division, enzymes involves in metabolic pathways, transcription factors and heat shock proteins.

22. A virus according to any one of claims 13 to 21 for use in delivering said heterologous gene to a mammalian cell.

23. A virus according to any one of claims 13 to 22 for use in a method of treatment of the human or animal body.

24. A virus according to claim 23 for use in the treatment of disorders of, or injuries to, the nervous system of a mammal.

25. Use of a herpes simplex virus according to any one of claims 13 to 22 in the manufacture of a medicament for use in the treatment of the human or animal body.

26. Use of a herpes simplex virus according to claim 25 in the treatment of disorders of, or injuries to, the nervous system of a mammal.

27. A pharmaceutical composition comprising an HSV strain according to any one of claims 13 to 22 together with a pharmaceutically acceptable carrier or diluent.

28. A method for studying the function of a heterologous gene in a mammalian cell which method comprises:
(a) introducing said heterologous gene into a herpes simplex virus according to any one of claims 1 to 12;
(b) introducing the resulting herpes simplex virus into said mammalian cell, and
(c) determining the effect of expression of said heterologous gene in said mammalian cell.

29. A method according to claim 28 wherein said heterologous gene is a wild-type or mutant gene implicated in causing disease.

30. A method according to claim 28 or 29 wherein said mammalian cell is dysfunctional, said heterologous gene is wild-type and the effect of expression of said heterologous gene is determined by an assay for cellular function.

31. A method according to claim 28 or 29 wherein said mammalian cell has one or more endogenous genes inactivated by mutation.

32. A method for producing a herpes simplex virus according to claim 1, said method comprising:
(i) inserted the HSV LAT sequence into an essential IE gene of the virus; and
(ii) deleting at least part of the LAT region of the virus.

33. A method of treating a disorder of, or injury to, the nervous system of a mammal comprising administering to a patient in need thereof an effective amount of a virus according to any one of claims 10 to 19.
